(19) United States
(12) Patent Application Publication
(10) Pub. No.: US 2013/0066130 A1
(43) Pub. Date: Mar. 14, 2013

(54) IONIC LIQUID CATALYZED ALKYLATION PROCESSES & SYSTEMS

(71) Applicants: Huping Luo, Richmond, CA (US); Moinuddin Ahmed, Hercules, CA (US)

(72) Inventors: Huping Luo, Richmond, CA (US); Moinuddin Ahmed, Hercules, CA (US)

(73) Assignee: Chevron U.S.A. Inc., San Ramon, CA (US)

(21) Appl. No.: 13/230,749

(22) Filed: Sep. 12, 2011

(51) Int. Cl. C07C 6/00; B01J 19/00

(52) U.S. Cl. 585/643; 422/187

(57) ABSTRACT

Ionic liquid alkylation processes may comprise contacting at least one hydrocarbon stream with an ionic liquid catalyst in an ionic liquid alkylation zone under ionic liquid alkylation conditions, cooling at least one of a reactor effluent and a hydrocarbon phase of the reactor effluent, and recycling the cooled reactor effluent or cooled hydrocarbon phase to the ionic liquid alkylation zone. Ionic liquid alkylation systems for performing ionic liquid catalyzed alkylation processes are also disclosed.

### TECHNICAL FIELD

The present invention relates to ionic liquid catalyzed alkylation processes and systems.

### BACKGROUND

Hydrofluoric acid (HF) is used as a catalyst in conventional industrial processes to perform reactions such as aromatic and olefin alkylation, including refinery processes for the production of high-octane gasoline, distillate, and lubricating base oil. The hazards of HF, e.g., related to HF volatility, are well documented. The use of additives to reduce HF volatility is expensive and does not eliminate the need for large quantities of HF in the plant.

Efforts to develop safer, alternative catalysts have encountered serious challenges. The conversion of HF alkylation units to use sulfuric acid (H₂SO₄) as catalyst requires significant added capital and operating expense, and at the same time introduces the hazards associated with highly corrosive concentrated H₂SO₄. Further, solid alkylation catalysts have proved difficult to commercialize due to rapid fouling and deactivation.

FIG. 1 schematically represents an HF alkylation unit 10, according to the prior art. HF alkylation unit 10 includes a hydrocarbon feed line 12, an HF reactor 16, and an HF settler 18 coupled to HF reactor 16 for separating an HF/hydrocarbon mixture into a hydrocarbon phase 20 and an HF phase 22. In conventional HF alkylation processes, a large excess of HF catalyst is used such that the HF forms the continuous liquid phase in which the hydrocarbon feeds are dispersed. Due to the relatively large volume of HF catalyst used in HF alkylation, reaction heat may be adequately dissipated by recycling the HF phase via heat exchanger 24. In FIG. 1, heat exchanger 24 is shown as being external to HF reactor 16. In other designs, the heat exchanger could be located inside the reactor to remove the reaction heat from both the hydrocarbon and the HF together.

The quest for alternative catalytic systems to replace conventional HF and H₂SO₄ catalysts in alkylation processes has been researched by various groups in both academic and industrial institutions. Thus far, no alternative catalyst for performing such processes has been commercialized.

Recently there has been considerable interest in metal halide ionic liquid catalysts as alternatives to HF and H₂SO₄ catalysts. As an example, the ionic liquid catalyzed alkylation of isoparaffins with olefins is disclosed in U.S. Pat. No. 7,432,408 to Timken, et al. Further, U.S. Pat. No. 7,572,943 to Elomari, et al. discloses the ionic liquid catalyzed oligomerization of olefins and the alkylation of the resulting oligomers(s) with isoparaffins to produce alkylated olefin oligomers.

There is a need for ionic liquid catalyzed alkylation processes and for ionic liquid alkylation systems for conducting such processes.

### SUMMARY

In an embodiment of the present invention there is provided an ionic liquid catalyzed alkylation process, comprising contacting at least one hydrocarbon stream with an ionic liquid catalyst in an ionic liquid alkylation zone under ionic liquid alkylation conditions, separating a reactor effluent from the ionic liquid alkylation zone into an ionic liquid phase and a hydrocarbon phase, cooling a first portion of the hydrocarbon phase to provide a cooled hydrocarbon phase, and recycling the cooled hydrocarbon phase to the ionic liquid alkylation zone.

In another embodiment there is provided an ionic liquid catalyzed alkylation process comprising contacting at least one hydrocarbon stream with an ionic liquid catalyst in an ionic liquid alkylation zone under ionic liquid alkylation conditions, cooling a first portion of a reactor effluent from the ionic liquid alkylation zone to provide a cooled reactor effluent, and recycling the cooled reactor effluent to the ionic liquid alkylation zone.

In another embodiment there is provided an ionic liquid alkylation system comprising an ionic liquid alkylation reactor; an ionic liquid/hydrocarbon injection unit, in fluid communication with the ionic liquid alkylation reactor, for injecting an ionic liquid/hydrocarbon mixture into the ionic liquid alkylation reactor; an ionic liquid/hydrocarbon separation unit, in fluid communication with the ionic liquid alkylation reactor, for receiving a reactor effluent from the ionic liquid alkylation reactor and for separating the reactor effluent into an ionic liquid phase and a hydrocarbon phase; and a heat exchanger, in fluid communication with the ionic liquid/hydrocarbon separation unit, for cooling at least a portion of the hydrocarbon phase to provide a cooled hydrocarbon phase, wherein the heat exchanger is in fluid communication with the ionic liquid/hydrocarbon injection unit for feeding the cooled hydrocarbon phase to the ionic liquid alkylation reactor.

In another embodiment there is provided an ionic liquid alkylation system comprising an ionic liquid alkylation reactor; an ionic liquid/hydrocarbon injection unit, in fluid communication with the ionic liquid alkylation reactor, for injecting an ionic liquid/hydrocarbon mixture into the ionic liquid alkylation reactor; a heat exchanger, in fluid communication with the ionic liquid alkylation reactor, for cooling a first portion of a reactor effluent from the ionic liquid alkylation reactor to provide a cooled reactor effluent; and an ionic liquid/hydrocarbon separation unit, in fluid communication with the ionic liquid alkylation reactor, for receiving a second portion of the reactor effluent from the ionic liquid alkylation reactor and for separating the second portion of the reactor effluent into a hydrocarbon phase and an ionic liquid phase.

As used herein, the terms “comprising” and “comprises” mean the inclusion of named elements or steps that are identified following those terms, but not necessarily excluding other unnamed elements or steps.

### BRIEF DESCRIPTION OF THE DRAWINGS

FIG. 1 schematically represents a hydrofluoric acid (HF) alkylation unit, according to the prior art.

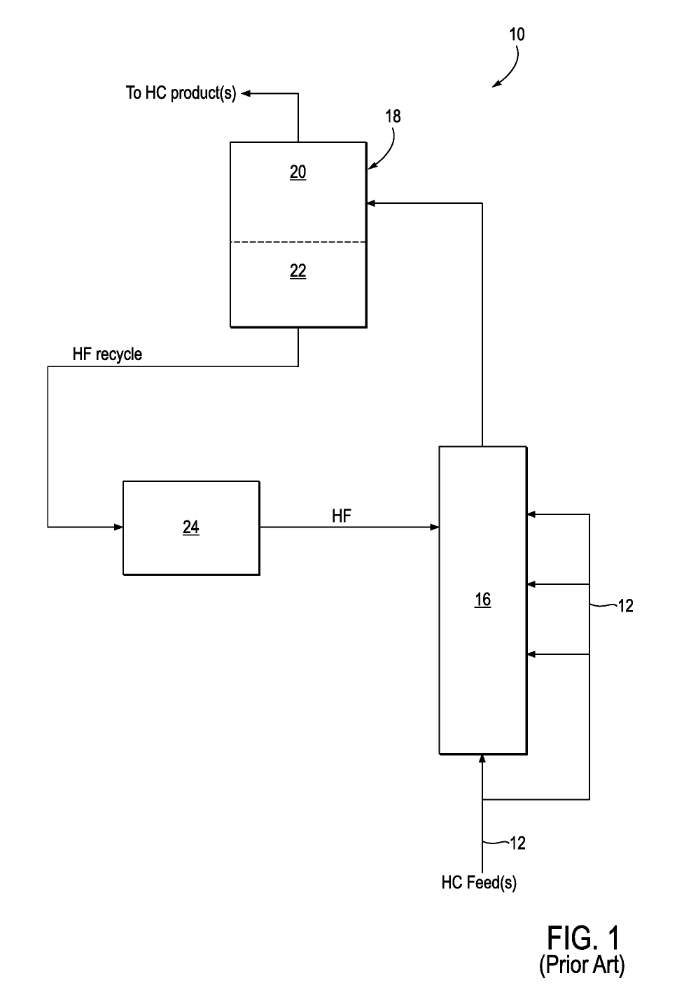

FIG. 2A schematically represents an ionic liquid alkylation system, according to an embodiment of the present invention;

FIG. 2B schematically represents an ionic liquid alkylation system, according to another embodiment of the present invention; and

FIGS. 3A and 3B each schematically represent an ionic liquid/hydrocarbon separation unit for the ionic liquid alkylation systems of FIGS. 2A and 2B, respectively.

### DETAILED DESCRIPTION

Ionic liquid catalysts may be useful for a range of hydrocarbon conversion reactions, including alkylation reactions for the production of alkylate gasoline blending components, distillate, lubricants, and the like. In an embodiment, the present invention provides ionic liquid catalyzed alkylation processes. In another embodiment, there is provided ionic liquid alkylation systems for efficiently performing ionic liquid catalyzed alkylation.

In ionic liquid catalyzed alkylation processes, the vol % of ionic liquid catalyst in the reactor is typically much less than that of HF catalyst used in an HF reactor of the prior art. Consequently, cooling the recycled ionic liquid catalyst phase alone may not adequately remove the excess heat generated during the ionic liquid catalyzed alkylation reaction. Instead, in embodiments of the instant invention, excess heat generated by the alkylation reaction may be effectively dissipated by cooling the recycled hydrocarbon phase or recycled reactor effluent.

Furthermore, the paraffin to olefin ratio of most hydrocarbon feeds may be considerably below the desired ratio for ionic liquid catalyzed alkylation for optimum product quality. According to embodiments of the invention, the paraffin to olefin ratio of the hydrocarbon stream introduced into the ionic liquid alkylation reactor may be effectively increased by recycling at least a portion of the alkylation reactor effluent or hydrocarbon phase to the ionic liquid alkylation reactor.

Ionic liquid catalyzed alkylation processes according to embodiments of the invention may comprise contacting at least one hydrocarbon stream with an ionic liquid catalyst in an ionic liquid alkylation reactor under ionic liquid alkylation conditions. In an embodiment, reactor effluent may be separated into an ionic liquid phase and a hydrocarbon phase, and the ionic liquid phase recycled to the ionic liquid alkylation reactor. A first portion of the hydrocarbon phase may be cooled and recycled to the ionic liquid alkylation reactor, while a second portion of the hydrocarbon phase may be fractionated to provide at least one hydrocarbon product. In another embodiment, a first portion of the reactor effluent may be cooled and recycled to the ionic liquid alkylation reactor, while a second portion of the reactor effluent may be phase separated. The ionic liquid phase may be recycled to the ionic liquid alkylation reactor, while the hydrocarbon phase may be fractionated to provide at least one hydrocarbon product.

### Feedstocks for Ionic Liquid Catalyzed Processes

In an embodiment, feeds for ionic liquid catalyzed hydrocarbon conversion processes may comprise various streams in a petroleum refinery, a gas-to-liquid conversion plant, a coal-to-liquid conversion plant, or in naphtha crackers, middle distillate crackers, or wax crackers, including FCC off-gas, FCC light naphtha, coker off-gas, coker naphtha, hydrocracker naphtha, and the like. In an embodiment, such streams may contain isoparaffin(s) and/or olefin(s).

Examples of olefin containing streams include FCC off-gas, coker gas, olefin metathesis unit off-gas, polyolefin gasoline unit off-gas, methanol to olefin unit off-gas, FCC light naphtha, coker light naphtha, Fischer-Tropsch unit condensate, and cracked naphtha.

Some olefin containing streams may contain two or more olefins selected from ethylene, propylene, butylenes, pentenes, and up to C₁₀ olefins. Such olefin containing streams are further described, for example, in U.S. Pat. No. 7,572,943, the disclosure of which is incorporated by reference herein in its entirety.

Examples of isoparaffin containing streams include, but are not limited to, FCC naphtha, hydrocracker naphtha, coker naphtha, Fisher-Tropsch unit condensate, and cracked naphtha. Such streams may comprise a mixture of two or more isoparaffins. In a sub-embodiment, an isoparaffin feed for an ionic liquid catalyzed process may comprise isobutane, which may be obtained, for example, from an n-butane isomerization plant, or a hydrocracking unit, or may be purchased.

In an embodiment, olefins and isoparaffins in the feed(s) may participate in ionic liquid catalyzed isoparaffin-olefin alkylation reactions. In another embodiment, olefins in the feed(s) may undergo oligomerization when contacted with an ionic liquid catalyst in a hydrocarbon conversion reactor. Ionic liquid catalyzed olefin oligomerization may take place under the same or similar conditions as ionic liquid catalyzed olefin-isoparaffin alkylation. Ionic liquid catalyzed olefin oligomerization and olefin-isoparaffin alkylation are disclosed, for example, in commonly assigned U.S. Pat. Nos. 7,572,943 and 7,576,252, the disclosures of which are incorporated by reference herein in their entirety.

### Ionic Liquid Catalysts

Ionic liquids are generally organic salts with melting points below 100° C. (212° F.) and often below room temperature. They may find applications in various chemical reactions, solvent processes, and electrochemistry. The use of chloroaluminate ionic liquids as alkylation catalysts in petroleum refining has been described, for example, in commonly assigned U.S. Pat. Nos. 7,531,707, 7,569,740, and 7,732,654, the disclosure of each of which is incorporated by reference herein in its entirety.

Most ionic liquids are prepared from organic cations and inorganic or organic anions. Cations include, but are not limited to, ammonium, phosphonium and sulphonium. Anions include, but are not limited to, BF₄⁻, PF₆⁻, haloaluminates such as Al₂Cl₇⁻ and Al₂Br₇⁻, [(CF₃SO₂)₂N]⁻, alkyl sulfates (RSO₃⁻), and carboxylates (RCO₂⁻). Ionic liquids for acid catalysis may include those derived from ammonium halides and Lewis acids, such as AlCl₃, TiCl₄, SnCl₄, and FeCl₃. Chloroaluminate ionic liquids are perhaps the most commonly used ionic liquid catalyst systems for acid catalyzed reactions.

Exemplary ionic liquids for use as catalysts in ionic liquid catalyzed alkylation reactions may comprise at least one compound of the general formulas A and B:

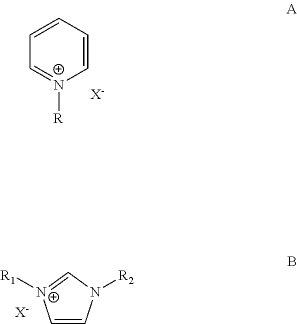

wherein R is H, methyl, ethyl, propyl, butyl, pentyl or hexyl; each of R₁ and R₂ is H, methyl, ethyl, propyl, butyl, pentyl or hexyl, wherein R₁ and R₂ may or may not be the same; and X is a chloroaluminate.

Non-limiting examples of chloroaluminate ionic liquid catalysts that may be used in alkylation processes according to embodiments of the instant invention include those comprising 1-butyl-4-methyl-pyridinium chloroaluminate, 1-butyl-3-methyl-imidazolium chloroaluminate, 1-H-pyridinium chloroaluminate, N-butylpyridinium chloroaluminate, and mixtures thereof.

### Systems for Ionic Liquid Catalyzed Alkylation

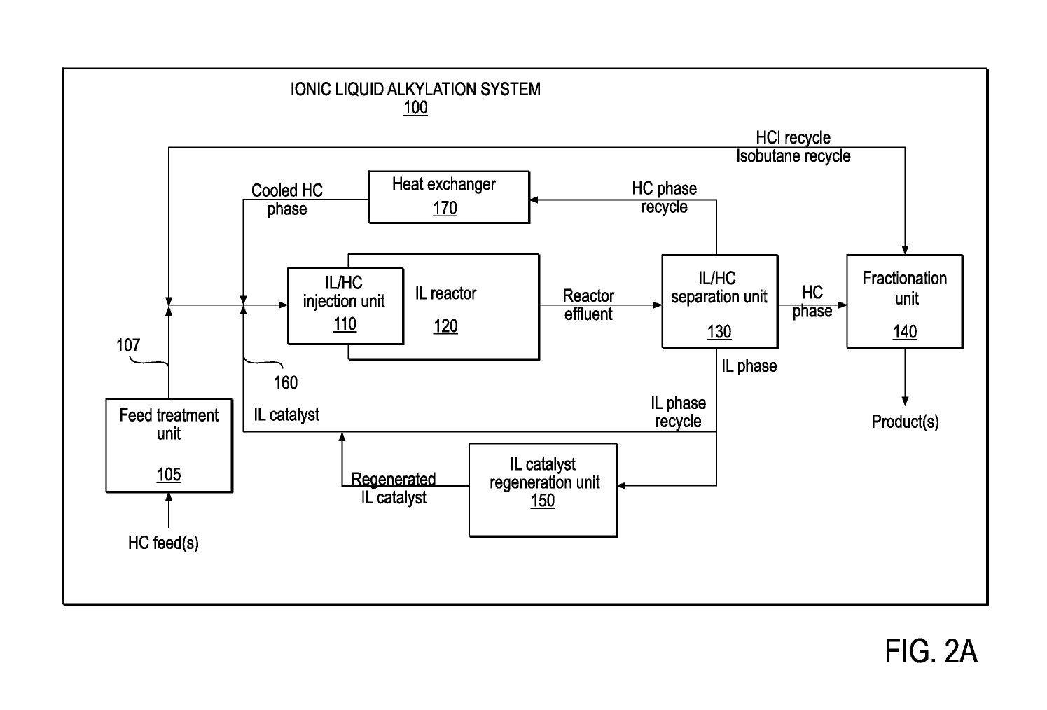

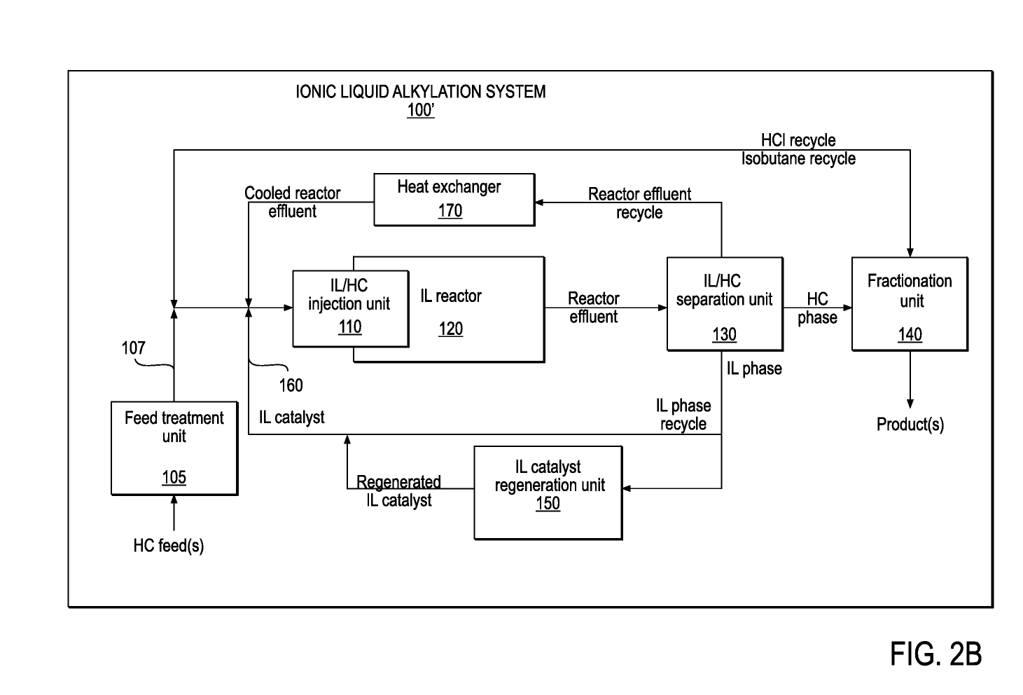

Embodiments of the present invention provide ionic liquid alkylation systems configured for efficiently performing ionic liquid alkylation processes. FIG. 2A schematically represents an ionic liquid (IL) alkylation system 100, according to an embodiment of the present invention. Ionic liquid alkylation system 100 may comprise a feed treatment unit 105, at least one hydrocarbon (HC) feed line 107, an ionic liquid/hydrocarbon injection unit 110, an ionic liquid alkylation reactor 120, an ionic liquid/hydrocarbon separation unit 130, a fractionation unit 140, an ionic liquid catalyst regeneration unit 150, an ionic liquid catalyst feed line 160, and a heat exchanger 170. Ionic liquid alkylation system 100 may also be referred to herein as ionic liquid catalyst alkylation system 100.

Feed treatment unit 105 may be configured for treating at least one hydrocarbon feed for ionic liquid catalyzed alkylation reactions. Feed treatment unit 105 may include one or more dryer units (not shown) for drying the hydrocarbon feeds, as well as one or more hydroisomerization units (also not shown) configured for the selective hydrogenation and hydroisomerization of olefin feeds. Feed treatment unit 105 may be in fluid communication with ionic liquid/hydrocarbon injection unit 110 via hydrocarbon feed line 107 for feeding at least one treated hydrocarbon feed to ionic liquid/hydrocarbon injection unit 110. Ionic liquid/hydrocarbon injection unit 110 may also be referred to herein as ionic liquid catalyst/hydrocarbon injection unit 110.

In an embodiment, ionic liquid/hydrocarbon injection unit 110 may be integral with ionic liquid alkylation reactor 120. In an embodiment, at least a portion, e.g., a distal portion, of ionic liquid/hydrocarbon injection unit 110 may extend into a void or space within ionic liquid alkylation reactor 120. In an embodiment, ionic liquid/hydrocarbon injection unit 110 may be disposed at least partially within ionic liquid alkylation reactor 120. In an embodiment, ionic liquid/hydrocarbon injection unit 110 may be a part or component of ionic liquid alkylation reactor 120.

In an embodiment, ionic liquid/hydrocarbon injection unit 110 may include at least one nozzle. Nozzles for introducing ionic liquid catalyst and hydrocarbon feeds into an ionic liquid alkylation reactor are disclosed in commonly assigned U.S. Patent Application Publication Nos. 20090166257, 20090171133, and 20090171134, and in U.S. patent application Ser. No. 12/780452, filed May 14, 2010, the disclosure of each of which is incorporated by reference herein in its entirety.

Ionic liquid/hydrocarbon injection unit 110 may be in fluid communication with ionic liquid alkylation reactor 120 for injecting an ionic liquid/hydrocarbon mixture into ionic liquid alkylation reactor 120. Ionic liquid reactor 120 may be configured for ionic liquid catalyzed alkylation reactions. Ionic liquid alkylation reactor 120 may also be referred to herein as ionic liquid reactor 120.

The ionic liquid/hydrocarbon mixture in ionic liquid reactor 120 may comprise an ionic liquid phase and a hydrocarbon phase. The ionic liquid phase may also be referred to herein as an ionic liquid catalyst phase. In an embodiment, ionic liquid reactor 120 and ionic liquid/hydrocarbon injection unit 110 may be co-configured for forming, in ionic liquid reactor 120, an emulsion comprising a dispersed ionic liquid phase and a continuous hydrocarbon phase. In an embodiment, droplets of ionic liquid catalyst may be uniformly distributed in the continuous hydrocarbon phase, and the emulsion in ionic liquid reactor 120 may be homogeneous. In some embodiments, other reactor configurations could be utilized for ionic liquid catalyzed alkylation to achieve a uniform homogeneous mixture of ionic liquid catalyst and hydrocarbon, such as an in-line mixer, or a continuous stirred tank reactor with one or more impellers.

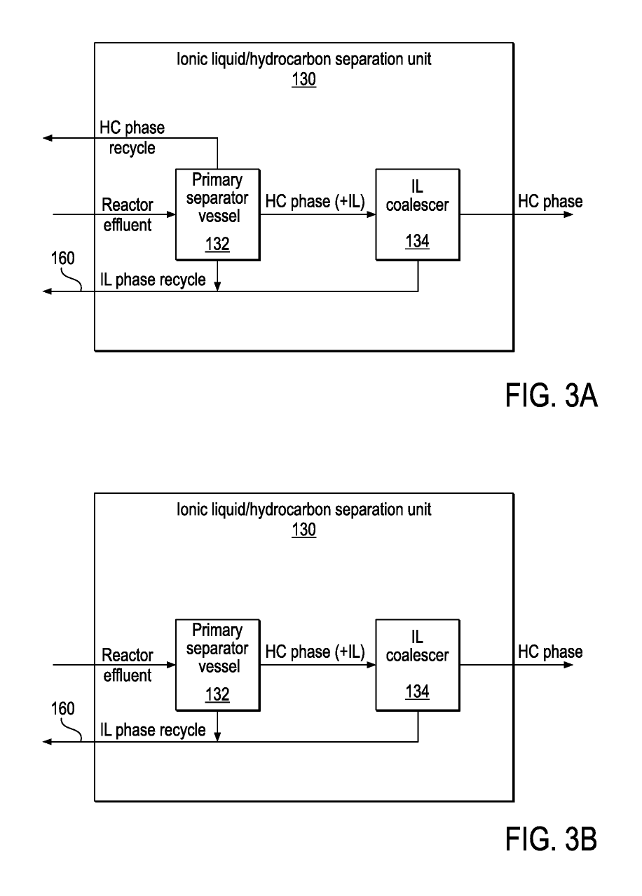

FIG. 3A schematically represents an ionic liquid/hydrocarbon separation unit for an ionic liquid catalyzed alkylation system, according to an embodiment of the present invention. Ionic liquid/hydrocarbon separation unit 130 may include a primary separator vessel 132, and an ionic liquid coalescer 134 in fluid communication with primary separator vessel 132. Ionic liquid/hydrocarbon separation unit 130 may also be referred to herein as ionic liquid catalyst/hydrocarbon separation unit 130.

With further reference to FIGS. 2A & 3A, ionic liquid/hydrocarbon separation unit 130 may be in fluid communication with ionic liquid reactor 120 via primary separator vessel 132. Primary separator vessel 132 may be configured for receiving alkylation reactor effluent from ionic liquid reactor 120 and for separating the reactor effluent into the hydrocarbon phase and the ionic liquid phase.

Primary separator vessel 132 may also be in fluid communication with heat exchanger 170 for feeding at least a first portion of the hydrocarbon phase from primary separator vessel 132 to heat exchanger 170. Heat exchanger 170 may be configured for cooling the first portion of the hydrocarbon phase to provide a cooled hydrocarbon phase.

Heat exchanger 170 may be in fluid communication with ionic liquid/hydrocarbon injection unit 110 for feeding or recycling the cooled hydrocarbon phase to ionic liquid alkylation reactor 120. The ionic liquid/hydrocarbon mixture may comprise, inter alia, the cooled hydrocarbon phase and an ionic liquid catalyst. Some exemplary ionic liquid catalysts are described hereinabove.

Ionic liquid/hydrocarbon separation unit 130 may also be in fluid communication with ionic liquid catalyst regeneration unit 150. In an embodiment, a first portion of the ionic liquid phase from primary separator vessel 132 may be recycled to ionic liquid reactor 120. A second portion of the ionic liquid phase from ionic liquid/hydrocarbon separation unit 130 may be fed to ionic liquid catalyst regeneration unit 150 for ionic liquid catalyst regeneration. Regenerated ionic liquid catalyst may be fed from regeneration unit 150 to ionic liquid alkylation reactor 120 via ionic liquid/hydrocarbon injection unit 110.

With still further reference to FIGS. 2A and 3A, ionic liquid coalescer 134 may be in fluid communication with primary separator vessel 132 for receiving a second portion of the hydrocarbon phase therefrom. Ionic liquid coalescer 134 may be configured for separating and removing (e.g., by coalescing) any ionic liquid entrained in the second portion of the hydrocarbon phase. The hydrocarbon phase from ionic liquid coalescer 134 may be at least substantially free from ionic liquid. Ionic liquid alkylation system 100 may be configured for recycling the coalesced ionic liquid from ionic liquid coalescer 134 to ionic liquid reactor 120. Ionic liquid coalescer 134 may also be referred to herein as ionic liquid catalyst coalescer 134.

Ionic liquid coalescer 134 may be in fluid communication with fractionation unit 140 for feeding the second portion of the hydrocarbon phase thereto. Fractionation unit 140 may be configured for fractionating the hydrocarbon phase to provide one or more hydrocarbon products, and for separating isobutane and HCl. The HCl may be separated primarily as an HCl-rich C_{3—} fraction. At least a portion of each of the isobutane and the HCl may be recycled to ionic liquid reactor 120. System 100 may further comprise a product treatment unit (not shown) for removing any contaminants from the hydrocarbon product(s).

FIG. 2B schematically represents an ionic liquid alkylation system 100′, according to another embodiment of the present invention. Ionic liquid alkylation system 100′ may comprise a feed treatment unit 105, at least one hydrocarbon feed line 107, an ionic liquid/hydrocarbon injection unit 110, an ionic liquid reactor 120, an ionic liquid/hydrocarbon separation unit 130, a fractionation unit 140, an ionic liquid catalyst regeneration unit 150, an ionic liquid catalyst feed line 160, and a heat exchanger 170, substantially as described hereinabove with reference to FIG. 2A.

In the embodiment of FIG. 2B, system 100′ may be configured for feeding a first portion of the reactor effluent from ionic liquid alkylation reactor 120 to heat exchanger 170 to provide cooled reactor effluent. Thus according to the embodiment of FIG. 2B, at least a portion of the alkylation reactor effluent may be cooled in the absence of ionic liquid/hydrocarbon phase separation. Heat exchanger 170 may be in fluid communication with ionic liquid/hydrocarbon injection unit 110 for recycling the cooled reactor effluent to ionic liquid reactor 120.

With reference to FIGS. 2B and 3B, system 100′ may also be configured for feeding a second portion of the reactor effluent from ionic liquid reactor 120 to ionic liquid/hydrocarbon separation unit 130, and for separating the second portion of the reactor effluent into the ionic liquid phase and the hydrocarbon phase. Otherwise, system 100′ may be configured at least substantially as described for system 100 of FIG. 2A.

In an alternative configuration (not shown) of a system for an ionic liquid catalyzed alkylation process according to another embodiment of the instant invention, the hydrocarbon and ionic liquid phases to be recycled to the ionic liquid reactor may be combined, and the combined phases cooled, e.g., via a heat exchanger. The cooled ionic liquid and hydrocarbon phases may then be recycled to the ionic liquid reactor, e.g., via one or more recycle pumps (not shown).

### Ionic Liquid Catalyzed Alkylation Processes

An ionic liquid catalyzed alkylation process according to an embodiment of the invention will now be described with reference to FIGS. 2A and 3A. During an ionic liquid catalyzed alkylation process conducted using system 100, an ionic liquid/hydrocarbon mixture may be introduced into ionic liquid reactor 120. In an embodiment, the ionic liquid/hydrocarbon mixture may be introduced into ionic liquid reactor 120 by injecting the mixture, e.g., via ionic liquid/hydrocarbon injection unit 110.

The ionic liquid/hydrocarbon mixture introduced into ionic liquid reactor 120 may comprise an ionic liquid catalyst and at least one hydrocarbon stream. The at least one hydrocarbon stream may comprise one or more treated hydrocarbon feed(s) from feed treatment unit 105. In an embodiment, the treated hydrocarbon feed(s) may comprise at least one C₄-C₁₀ isoparaffin and at least one C₂-C₁₀ olefin. Treatment of the hydrocarbon feed(s) may include feed drying, as well as the removal of dienes and the hydroisomerization of olefins in olefin feeds.

The at least one hydrocarbon stream introduced into ionic liquid reactor 120 may further comprise recycled isoparaffin(s), e.g., isobutane, from fractionation unit 140. The at least one hydrocarbon stream may still further comprise at least a portion of the hydrocarbon phase from ionic liquid/hydrocarbon separation unit 130, wherein the hydrocarbon phase may be cooled and then recycled to ionic liquid reactor 120. The cooled hydrocarbon phase may comprise, for example, unreacted isoparaffins and alkylate product.

The at least one hydrocarbon stream may be contacted with the ionic liquid catalyst in ionic liquid reactor 120 under ionic liquid alkylation conditions. Ionic liquid reactor 120 may also be referred to herein as an ionic liquid alkylation zone. In an embodiment, the ionic liquid catalyst may comprise a chloroaluminate ionic liquid, such as a compound of the general formulas A and B, supra. A co-catalyst such as anhydrous HCl, and/or a catalyst promoter such as an alkyl chloride, may also be fed to ionic liquid reactor 120. Exemplary reaction conditions for ionic liquid alkylation according to embodiments of the present invention are described hereinbelow.

With further reference to FIGS. 2A and 3A, reactor effluent from ionic liquid alkylation reactor 120 may be fed to primary separator vessel 132 of ionic liquid/hydrocarbon separation unit 130. The reactor effluent may be separated by primary separator vessel 132 into an ionic liquid phase and a hydrocarbon phase. At least a first portion of the hydrocarbon phase from primary separator vessel 132 may be cooled to provide a cooled hydrocarbon phase. In an embodiment, the first portion of the hydrocarbon phase may be cooled, for example, to a temperature in the range from 0-100° C. (32-212° F.), by passage through heat exchanger 170.

In an embodiment, the first portion of the hydrocarbon phase may comprise 5-95 vol % of the total hydrocarbon phase fed to primary separator vessel 132. In a sub-embodiment, the first portion of the hydrocarbon phase may comprise 50-95 vol % of the total hydrocarbon phase fed to primary separator vessel 132.

The cooled hydrocarbon phase may be recycled to ionic liquid reactor 120. In an embodiment, the cooled hydrocarbon phase may be recycled from heat exchanger 170 to ionic liquid reactor 120 via ionic liquid/hydrocarbon injection unit 110. In an embodiment, the recycle rate of the cooled hydrocarbon phase to ionic liquid reactor 120 may be varied, for example, to effectively remove the heat generated during the ionic liquid catalyzed alkylation reaction.

At least a portion of the ionic liquid phase from ionic liquid/hydrocarbon separation unit 130 may also be recycled to ionic liquid reactor 120. In an embodiment, the ionic liquid phase may be recycled from primary separator vessel 132 to ionic liquid reactor 120 via ionic liquid feed line 160. The recycled ionic liquid phase may be fed, together with the cooled hydrocarbon phase, to ionic liquid/hydrocarbon injection unit 110 for introduction into ionic liquid reactor 120.

With continued operation of system 100, the ionic liquid catalyst may become at least partially deactivated. In order to maintain catalytic activity of the ionic liquid, a portion of the ionic liquid phase from ionic liquid/hydrocarbon separation unit 130 may be fed to ionic liquid catalyst regeneration unit 150 for regeneration of the ionic liquid catalyst. Thereafter, the regenerated ionic liquid catalyst may be recycled to ionic liquid reactor 120, e.g., via ionic liquid feed line 160. The regeneration of ionic liquid catalysts is disclosed, for example, in commonly assigned U.S. Pat. Nos. 7,674,739, 7,955,999 and 7,956,002, the disclosure of each of which is incorporated by reference herein in its entirety.

With still further reference to FIGS. 2A and 3A, a second portion of the hydrocarbon phase may be fed from primary separator vessel 132 to ionic liquid coalescer 134. In an embodiment, the hydrocarbon phase may comprise some (e.g., trace amounts of) ionic liquid catalyst. As a non-limiting example, droplets of ionic liquid catalyst may be entrained in the hydrocarbon phase from primary separator vessel 132. Any such ionic liquid catalyst in the hydrocarbon phase may be separated, e.g., coalesced into larger droplets, and removed from the hydrocarbon phase by ionic liquid coalescer 134.

The hydrocarbon phase obtained from ionic liquid coalescer 134 may be at least substantially free from ionic liquid catalyst. The use of coalescers for liquid-liquid separations is described in commonly assigned U.S. Patent Application Publication No. 20100130800, the disclosure of which is incorporated by reference herein in its entirety. The ionic liquid removed or coalesced from the second portion of the hydrocarbon phase by ionic liquid coalescer 134 may be recycled to ionic liquid alkylation reactor 120.

The hydrocarbon phase from ionic liquid coalescer 134 may be fractionated, e.g., via fractionation unit 140, to provide one or more hydrocarbon products. The at least one hydrocarbon product may comprise, for example, alkylate gasoline, diesel fuel, jet fuel, base oil, and combinations thereof. Fractionation of the hydrocarbon phase may also yield isobutane and an HCl-rich C_{3—} fraction. Both the isobutane and HCl-rich fraction may be recycled to ionic liquid reactor 120. In an embodiment, the isobutane may be recycled to ionic liquid reactor 120 via ionic liquid/hydrocarbon injection unit 110.

An ionic liquid catalyzed alkylation process according to another embodiment of the invention will now be described with reference to FIGS. 2B and 3B. During an ionic liquid catalyzed alkylation process conducted using system 100′, an ionic liquid/hydrocarbon mixture comprising at least one hydrocarbon stream and an ionic liquid catalyst may be introduced into ionic liquid reactor 120. The at least one hydrocarbon stream may be contacted with the ionic liquid catalyst in ionic liquid reactor 120 under ionic liquid catalyzed alkylation conditions to provide at least one alkylate product. Ionic liquid reactor 120 of FIG. 2B may also be referred to herein as an ionic liquid alkylation zone.

A first portion of the reactor effluent from ionic liquid reactor 120 may be fed to heat exchanger 170 to provide cooled reactor effluent. The cooled reactor effluent may be recycled to ionic liquid reactor 120. In an embodiment, the first portion of the reactor effluent cooled via heat exchanger 170 may comprise 5-95 vol % of the total reactor effluent from ionic liquid reactor 120. In a sub-embodiment, the first portion of the reactor effluent cooled via heat exchanger 170 may comprise 50-95 vol % of the total reactor effluent from ionic liquid reactor 120. In an embodiment, the first portion of the reactor effluent may be cooled, for example, to a temperature in the range from 0-100° C., by passage through heat exchanger 170.

In an embodiment, the at least one hydrocarbon stream introduced into ionic liquid reactor 120 may comprise one or more treated hydrocarbon feeds from feed treatment unit 105. The at least one hydrocarbon stream may further comprise alkylate product and/or unreacted hydrocarbons in the cooled reactor effluent. The at least one hydrocarbon stream may still further comprise recycled isobutane from fractionation unit 140.

With further reference to FIGS. 2B and 3B, a second portion of the reactor effluent may be fed to ionic liquid/hydrocarbon separation unit 130. The second portion of the reactor effluent may be separated by ionic liquid/hydrocarbon separation unit 130 to provide an ionic liquid phase and a hydrocarbon phase. The ionic liquid phase may be processed, e.g., regenerated and/or recycled, substantially as for the alkylation process described hereinabove with reference to FIG. 2A.

The hydrocarbon phase may be fractionated, e.g., via fractionation unit 140, to provide isobutane, an HCl-rich fraction, and at least one hydrocarbon product, substantially as described hereinabove with reference to FIG. 2A. The at least one hydrocarbon product may be selected from, for example, alkylate gasoline, diesel fuel, jet fuel, base oil, and combinations thereof. Prior to fractionation, any entrained ionic liquid in the hydrocarbon phase may be separated and removed therefrom, e.g., entrained ionic liquid may be coalesced from the hydrocarbon phase via ionic liquid coalescer 134.

### Reaction Conditions for Ionic Liquid Catalyzed Processes

Due to the low solubility of hydrocarbons in ionic liquids, hydrocarbon conversion reactions in ionic liquids (including isoparaffin-olefin alkylation reactions) are generally biphasic and occur at the interface in the liquid state. The volume of ionic liquid catalyst in the reactor may be generally in the range from about 1 to 70 vol %, and usually from about 3 to 40 vol %. In an embodiment, an ionic liquid/hydrocarbon injection unit may be used to co-inject reactant(s) and ionic liquid catalyst into the ionic liquid alkylation reactor to ensure good contact between the ionic liquid catalyst and reactants.

The alkylation reaction temperature may be generally in the range from about −40 to +250° C. (−40 to +482° F.), typically from about −20 to +100° C. (−4 to +212° F.), and often from about +4 to +60° C. (+40 to +140° F.). The alkylation reactor pressure may be in the range from atmospheric pressure to about 8000 kPa. Typically, the reactor pressure is sufficient to keep the reactants in the liquid phase.

Residence time of reactants in the alkylation reactor may generally be in the range from a few seconds to hours, and usually from about 0.5 min to 60 min. In the case of ionic liquid catalyzed isoparaffin-olefin alkylation, a hydrocarbon stream introduced into the alkylation reactor may have an isoparaffin:olefin molar ratio generally in the range from about 1-200, more typically from about 2-100, and often from about 5-50. Reactor conditions may be adjusted to optimize process performance for a particular ionic liquid catalyzed alkylation process or targeted product(s).

There are numerous variations on the present invention which are possible in light of the teachings and supporting examples described herein. It is therefore understood that within the scope of the following claims, the invention may be practiced otherwise than as specifically described or exemplified herein.

What is claimed is:

1. An ionic liquid catalyzed alkylation process, comprising:
a) contacting at least one hydrocarbon stream with an ionic liquid catalyst in an ionic liquid alkylation zone under ionic liquid alkylation conditions;
b) separating a reactor effluent from the ionic liquid alkylation zone into an ionic liquid phase and a hydrocarbon phase;
c) cooling a first portion of the hydrocarbon phase to provide a cooled hydrocarbon phase; and
d) recycling the cooled hydrocarbon phase to the ionic liquid alkylation zone.

2. The process according to claim 1, further comprising:
e) recycling the ionic liquid phase to the ionic liquid alkylation zone.

3. The process according to claim 2, further comprising:
f) feeding an ionic liquid/hydrocarbon mixture to an ionic liquid/hydrocarbon injection unit, wherein the ionic liquid/hydrocarbon mixture comprises the cooled hydrocarbon phase and the ionic liquid phase.

4. The process according to claim 3, further comprising:
g) injecting, via the ionic liquid/hydrocarbon injection unit, the ionic liquid/hydrocarbon mixture into the ionic liquid alkylation zone.

5. The process according to claim 1, further comprising:
h) removing entrained ionic liquid from a second portion of the hydrocarbon phase; and
i) fractionating the second portion of the hydrocarbon phase to provide at least one hydrocarbon product.

6. The process according to claim 1, wherein the at least one hydrocarbon stream comprises at least one C₄-C₁₀ isoparaffin and at least one C₂-C₁₀ olefin, and the ionic liquid alkylation conditions comprise a reaction temperature in the range from about −20° C. to +100° C., a reaction pressure in the range from atmospheric pressure to 8000 kPa, and an isoparaffin:olefin molar ratio in the range from about 2-100.

7. The process according to claim 5, wherein the at least one hydrocarbon product is selected from the group consisting of alkylate gasoline, diesel fuel, jet fuel, base oil, and combinations thereof.

8. The process according to claim 1, wherein the ionic liquid catalyst comprises at least one compound of the general formulas A and B: 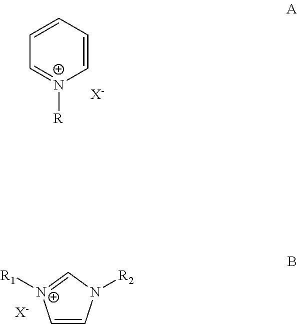
wherein R is selected from the group consisting of H, methyl, ethyl, propyl, butyl, pentyl and hexyl; each of R₁ and R₂ is selected from the group consisting of H, methyl, ethyl, propyl, butyl, pentyl and hexyl, wherein R₁ and R₂ may or may not be the same; and X is a chloroaluminate.

9. An ionic liquid catalyzed alkylation process, comprising:
a) contacting at least one hydrocarbon stream with an ionic liquid catalyst in an ionic liquid alkylation zone under ionic liquid alkylation conditions;
b) cooling a first portion of a reactor effluent from the ionic liquid alkylation zone to provide a cooled reactor effluent; and
c) recycling the cooled reactor effluent to the ionic liquid alkylation zone.

10. The process according to claim 9, further comprising:
d) separating a second portion of the reactor effluent into an ionic liquid phase and a hydrocarbon phase; and
e) fractionating the hydrocarbon phase to provide at least one hydrocarbon product.

11. The process according to claim 9, further comprising:
f) separating a second portion of the reactor effluent into an ionic liquid phase and a hydrocarbon phase; and
g) recycling the ionic liquid phase to the ionic liquid alkylation zone.

12. The process according to claim 9, further comprising:
h) feeding an ionic liquid/hydrocarbon mixture to an ionic liquid/hydrocarbon injection unit, wherein the ionic liquid/hydrocarbon mixture comprises the cooled reactor effluent.

13. The process according to claim 12, further comprising:
i) injecting, via the ionic liquid/hydrocarbon injection unit, the ionic liquid/hydrocarbon mixture into the ionic liquid alkylation zone.

14. The process according to claim 10, further comprising:
j) prior to step e), removing entrained ionic liquid from the hydrocarbon phase.

15. The process according to claim 9, wherein the at least one hydrocarbon stream comprises at least one C₄-C₁₀ isoparaffin and at least one C₂-C₁₀ olefin, and the ionic liquid alkylation conditions comprise a reaction temperature in the range from about −20° C. to +100° C., a reaction pressure in the range from atmospheric pressure to 8000 kPa, and an isoparaffin:olefin molar ratio in the range from about 2-100.

16. The process according to claim 10, wherein the at least one hydrocarbon product is selected from the group consisting of alkylate gasoline, diesel fuel, jet fuel, base oil, and combinations thereof.

17. The process according to claim 9, wherein the ionic liquid catalyst comprises at least one compound of the general formulas A and B: 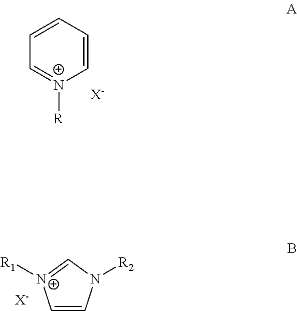
wherein R is selected from the group consisting of H, methyl, ethyl, propyl, butyl, pentyl and hexyl; each of R₁ and R₂ is selected from the group consisting of H, methyl, ethyl, propyl, butyl, pentyl and hexyl, wherein R₁ and R₂ may or may not be the same; and X is a chloroaluminate.

18. An ionic liquid alkylation system, comprising:
an ionic liquid alkylation reactor;
an ionic liquid/hydrocarbon injection unit, in fluid communication with the ionic liquid alkylation reactor, for injecting an ionic liquid/hydrocarbon mixture into the ionic liquid alkylation reactor;
an ionic liquid/hydrocarbon separation unit, in fluid communication with the ionic liquid alkylation reactor, for receiving a reactor effluent from the ionic liquid alkylation reactor and for separating the reactor effluent into an ionic liquid phase and a hydrocarbon phase; and
a heat exchanger, in fluid communication with the ionic liquid/hydrocarbon separation unit, for cooling at least a portion of the hydrocarbon phase to provide a cooled hydrocarbon phase, wherein the heat exchanger is in fluid communication with the ionic liquid/hydrocarbon injection unit for feeding the cooled hydrocarbon phase to the ionic liquid alkylation reactor.

19. The system according to claim 18, wherein the ionic liquid alkylation reactor and the ionic liquid/hydrocarbon injection unit are co-configured for forming, in the ionic liquid alkylation reactor, an emulsion comprising a dispersed ionic liquid phase and a continuous hydrocarbon phase.

20. The system according to claim 18, wherein:
the ionic liquid/hydrocarbon separation unit comprises a primary separator vessel and an ionic liquid coalescer in fluid communication with the primary separator vessel; and
the system is configured for recycling coalesced ionic liquid from the ionic liquid coalescer to the ionic liquid alkylation reactor.

21. An ionic liquid alkylation system, comprising:
an ionic liquid alkylation reactor;
an ionic liquid/hydrocarbon injection unit, in fluid communication with the ionic liquid alkylation reactor, for injecting an ionic liquid/hydrocarbon mixture into the ionic liquid alkylation reactor;
a heat exchanger, in fluid communication with the ionic liquid alkylation reactor, for cooling a first portion of a reactor effluent from the ionic liquid alkylation reactor to provide a cooled reactor effluent; and
an ionic liquid/hydrocarbon separation unit, in fluid communication with the ionic liquid alkylation reactor, for receiving a second portion of the reactor effluent from the ionic liquid alkylation reactor and for separating the second portion of the reactor effluent into a hydrocarbon phase and an ionic liquid phase.

22. The system according to claim 21, wherein:
the ionic liquid/hydrocarbon separation unit comprises a primary separator vessel and an ionic liquid coalescer in fluid communication with the primary separator vessel; and
the system is configured for recycling coalesced ionic liquid from the ionic liquid coalescer to the ionic liquid alkylation reactor.

23. The system according to claim 21, wherein the ionic liquid alkylation reactor and the ionic liquid/hydrocarbon injection unit are co-configured for forming, in the ionic liquid alkylation reactor, an emulsion comprising a dispersed ionic liquid phase and a continuous hydrocarbon phase.
